(19) United States
(12) Patent Application Publication
(10) Pub. No.: US 2008/0269804 A1
(43) Pub. Date: Oct. 30, 2008

(54) Apparatus and method for flexible spinal fixation

(75) Inventor: Richard T. Holt, Prospect, KY (US)

Correspondence Address: NIXON & VANDERHYE, PC, 901 NORTH GLEBE ROAD, 11TH FLOOR, ARLINGTON, VA, 22203, US

(73) Assignee: Holt Development L.L.C., Louisville, KY (US)

(21) Appl. No.: 12/149,397

(22) Filed: Apr. 30, 2008

Related U.S. Application Data
(63) Continuation-in-part of application No. 11/812,363, filed on Jun. 18, 2007.
Application No. 11/812,363 is a continuation-in-part of application No. 11/356,292, filed on Feb. 17, 2006.

(51) Int. Cl. A61B 17/70

(52) U.S. Cl. 606/254; 606/246; 606/250; 606/278

(57) ABSTRACT

Apparatus for connecting and stabilizing adjacent vertebral segments, comprising a flexible composite connecting rod extending between the segments, and connection devices for connecting the rod to the vertebral segments. The rod comprises a rod member formed of a flexible plastic material having a predetermined compression strength, and a high tensile strength, low stretch, flexible reinforcing element extending longitudinally through the entire length of the rod member. The reinforcing element may be in the form of a single cord, rope, braid or monofilament, a plurality of substantially parallel cords, ropes, braids or monofilaments, or a tubular cord, rope, braid or woven or wire mesh strain relief device extending through the rod member in slidable relation or bonded thereto.

### CROSS REFERENCE TO RELATED APPLICATIONS

This application is a continuation-in-part of application Ser. No. 11/812,363 filed on Jun. 18, 2007, which is a continuation-in-part of application Ser. No. 11/356,292 filed on Feb. 17, 2006.

### FIELD OF THE INVENTION

The present invention relates to an apparatus and method for spinal fixation and, more particularly, to such an apparatus and method for flexible stabilization of a vertebral column or the like.

### DESCRIPTION OF THE RELATED PRIOR ART

Stabilization of vertebral columns by instrumentation devices and/or bone material to facilitate a bone fusion is a common and long practiced surgical technique. Fusion is the permanent internal fixation of part or all of the intervertebral joints, an inter-vertebral joint being composed of two adjacent vertebrae and their posterior bony elements connected by an intervertebral disc, ligaments, and two facet joint capsules. It has been found that the use of fusion in many cases results in significant patient disability. By fusing vertebrae, the remaining segments are subject to inordinately high stress and degeneration.

When spine stabilization involves mechanical instrumentation, significant forces are directly aimed at the supportive sites whether they be bone screws, hooks or the like. This phenomenon usually produces loosening of the points of attachment for the implanted hardware and a resulting loss of support by this instrumentation unless fusion occurs. Because of this, stabilizations involving instrumentation are often carried out in connection with a bone fusion so that, as the instrumentation loosens and fails, support can be maintained by growth of the bony counterpart. These combined procedures involve extensive surgery, substantial blood loss and high costs. Following such a procedure, patients are usually disabled for long periods of time.

Spinal fixation systems utilizing polyaxial pedicle screws connecting metal rods or metal plates screwed to bone are the current standard for spinal fixation. These rigid devices hold the vertebrae in a fixed position to allow fusion to take place between the adjoining vertebral segments. This substantially rigid design makes perfect alignment nearly impossible and resulting stresses are passed to the bone. The surgeon must bend and manipulate the rigid components for the best possible alignment, which is difficult and time consuming and can result in decreased fatigue strength of the deformed metal. The stress imparted on components in addition to stresses applied by patient movement can lead to fatigue failure of metal components.

Recent innovations have utilized a polyurethane tube for compression resistance, and a polyethylene rope slidable within the tube to tension the tube between rigid pedicle screws. The small amount of controlled motion allowed by this approach has improved results for patients, has promoted healing and on occasion obviated the need for fusion. However, this system is difficult to align and tension, and requires cutting the tube to length during the surgical procedure.

A need has arisen, therefore, for a new and improved apparatus and method for flexible stabilization of a vertebral column or the like.

### SUMMARY OF THE INVENTION

The spinal fixation apparatus and method of the present invention serves to connect and stabilize adjacent vertebral segments to facilitate fusion procedures and/or to promote healing from trauma, disease or arthritic conditions. The new and improved apparatus of the present invention comprises one or more flexible composite connecting rods that are connected to the vertebral segments by any suitable spine implants or connectors, such as pedicle screws, vertebral screws or hook systems.

The flexible composite connecting rod comprises a rod member formed of a suitable, flexible, biocompatible material, such as polyurethane, UHMW polyethylene, PEEK or Teflon, having a desired compression strength. A high tensile strength, low stretch, flexible, biocompatible reinforcing element in, e.g., cord or fabric form is encased within and may be slidable in or bonded to the rod member, and extends longitudinally through the entire length thereof. A single large cord, multiple cords, a woven tube or wire mesh strain relief device, or the like may be used as the reinforcing element which may formed of any suitable material, such as Kevlar, polyethylene, polyurethane, Teflon fiber, carbon fiber or stainless steel. The composite connecting rod may be constructed to provide varying degrees of flexibility depending on the particular patient application.

As an illustrative embodiment, the composite connecting rod can be attached to adjacent vertebral segments by using polyaxial pedicle screws with a formed compression plate or pad riding under a set screw that is constructed to engage shoulder or stop portions in the open head portion of the pedicle screw to provide for controlled compression of the portion of the composite connecting rod inserted therein, and a controlled tightening torque to assure a positive lock for the set screw.

Alternatively, one or more compression rings may be mounted, crimped or press-fitted on the composite rod at predetermined locations so that the rings are received in the open head portions of the pedicle screws or the like. Each compression ring can be formed with a spherical or curved outer surface for self alignment within a complementary curved opening in the head portion of each pedicle screw, or may have a square or rectangular outer surface for rigid retention in complementary openings in the head portions of the pedicle screws. In one embodiment, split-compression rings are used which are compressed into engagement with the composite rods at predetermined locations.

### BRIEF DESCRIPTION OF THE DRAWINGS

FIG. 1 is a top elevation view of one embodiment of the apparatus of the present invention connected to adjacent vertebral segments for flexible stabilization of a vertebral column;

FIG. 2 is an exploded perspective view of the one embodiment of the spinal fixation apparatus shown in FIG. 1;

FIG. 3 is an exploded perspective view of another embodiment of the spinal fixation apparatus of the present invention;

FIG. 4 is a perspective view of a further embodiment of the spinal fixation apparatus of the present invention;

FIG. 5a is an exploded perspective view of the embodiment of the spinal fixation apparatus shown in FIG. 4;

FIG. 5b is a perspective view of a modified connecting rod similar to that shown in FIG. 5a:

FIG. 6 is a perspective view, with parts broken away, of a first embodiment of the flexible composite connecting rod of the present invention;

FIG. 7 is a perspective view, with parts broken away, of a second embodiment of the flexible composite connecting rod of the present invention;

FIG. 8 is a perspective view, with parts broken away, of a third embodiment of the flexible composite connecting rod of the present invention.

FIG. 9 is a perspective view of a further embodiment of a compression ring that can be mounted on the flexible connecting rod of the present invention;

FIG. 10 is an exploded perspective view of a further embodiment of the spinal fixation apparatus that can be used with the compression ring shown in FIG. 9; and

FIGS. 11a and 11b are perspective views of fourth and fifth embodiments, respectively, of the flexible composite connecting rod of the present invention.

### DETAILED DESCRIPTION OF THE PREFERRED EMBODIMENTS

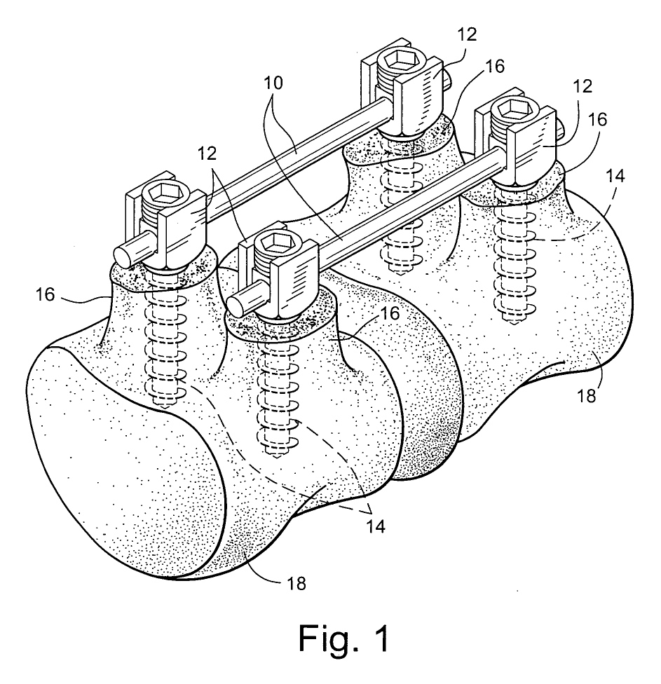

FIG. 1 illustrates an example of the use of the apparatus and method of the present invention wherein a pair of flexible composite connecting rods 10 of the present invention are secured to the head portions 12 of bone connection devices such as screws 14 or the like that are connected to the pedicle portions 16 of adjacent vertebral segments 18 for flexible stabilization of the vertebral segments in a desired manner.

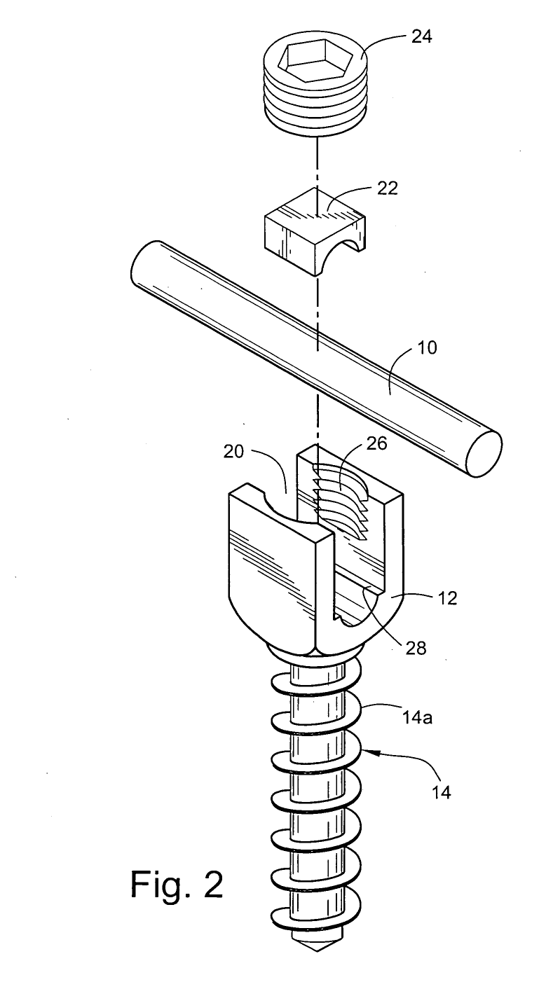

As shown in FIGS. 1 and 2, the screw 14 may be a polyaxial pedicle screw which has a head portion 12 and a screw portion 14a that are pivotally or movably connected together. The head portion 12 comprises an upper opening 20 that is shaped to receive the flexible, composite connecting rod 10 constructed in accordance with the principles of the present invention. A clamping pad 22 is received in the opening 20 of the head portion 12 and has a shape on the inner surface thereof that is complementary to the exterior shape of the flexible composite connecting rod 10. A set screw 24 is received in the upper threaded area 26 of the head portion 12 and serves to retain the clamping pad 22 in engagement with an internal shoulder or stop 28 in the head portion for the purpose of applying a predetermined compression on the flexible composite connecting rod 10 to retain it in position on the pedicle screw 14.

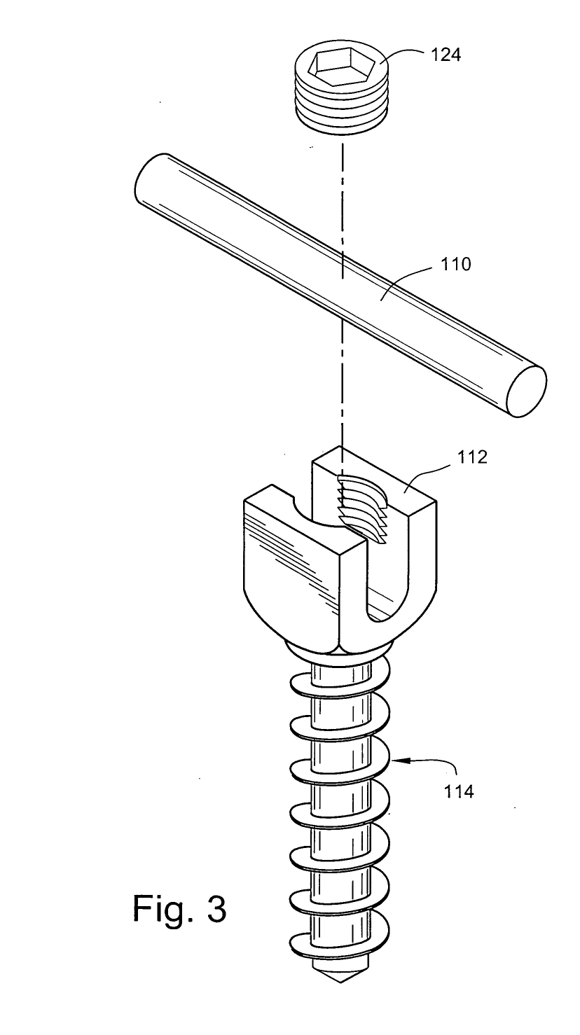

Alternatively, as shown in FIG. 3, the head portion 112 of the pedicle screw 114 may comprise a set screw 124 that engages the flexible composite connecting rod 110 directly without the use of a clamping pad to retain it in position on the pedicle screw.

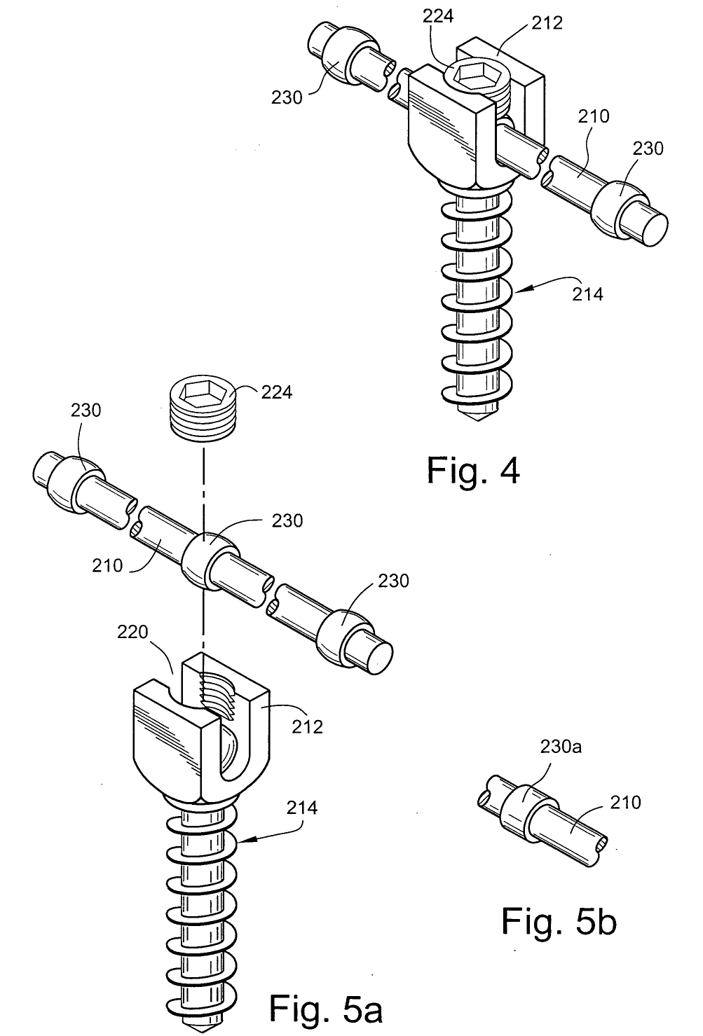

Referring to FIGS. 4, 5a and 5b, in a further embodiment, the flexible composite connecting rod 210 may have ring members 230, 230a fixedly mounted thereon at predetermined locations so as to be receivable in complementary openings 220 in the head portions 212 of pedicle screws 214 for engagement by set screws 224 or the like to retain the flexible composite connecting rod 210 on the pedicle screws. As an illustrative embodiment, the ring members 230, 230a may be made of a suitable metal or other material and press-fitted or crimped on the flexible composite connecting rod 210.

As shown in FIGS. 5a and 5b, the ring members 230 may have a spherical or curved shape for self alignment within the head portions 212 of the pedicle screw 214, or the ring members 230a may have a square or rectangular exterior shape for rigid alignment in the complementary openings 220 in the head portions of the pedicle screws.

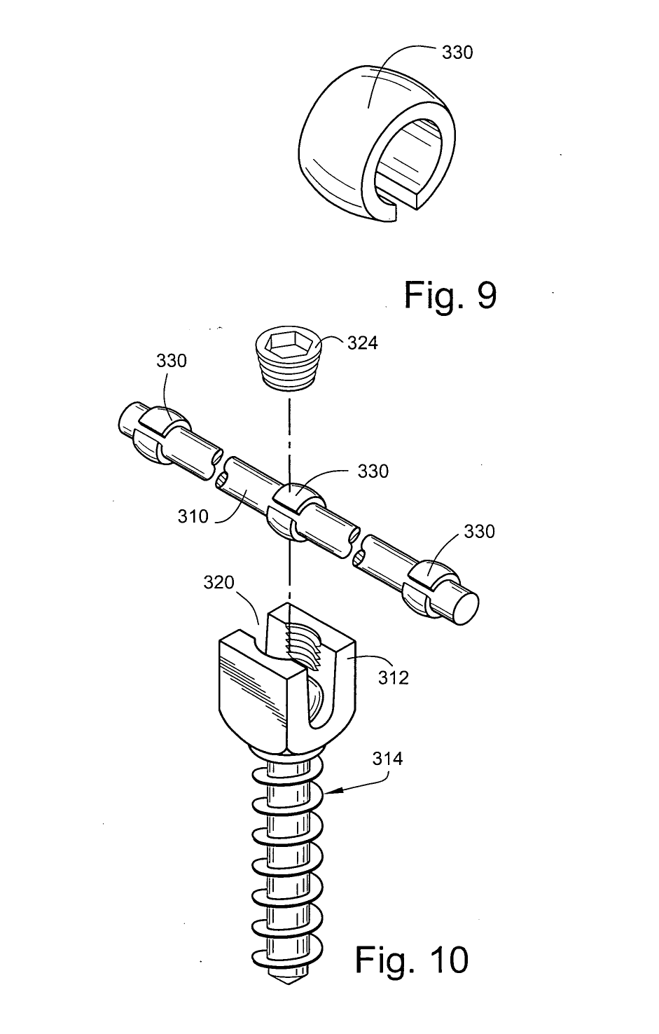

Referring to FIGS. 9 and 10, modified compression ring members 330 of split construction and of any suitable material may be slidably mounted on the flexible composite connecting rod 10 at predetermined locations so as to be receivable in complementary openings 320 in the head portions 312 of pedicle screws 314 for engagement by set screws 324 or the like to compress the compression ring members 330 into tight engagement with the connecting rod 310 to retain it on the pedicle screws 314. As shown in FIG. 10, the set screw 324 and openings 320 in the head portions 312 may be tapered downwardly and inwardly in a complementary manner to facilitate the compression of the split compression ring members 330 into tight engagement with the connecting rod 310. The ring members 330 may have a spherical or curved shape as shown in FIGS. 5a and 9, or may have a square or rectangular exterior shape like that shown in FIG. 5b for the ring member 230a. As an illustrative example, the ring members 330 may be formed of a suitable metal or other material.

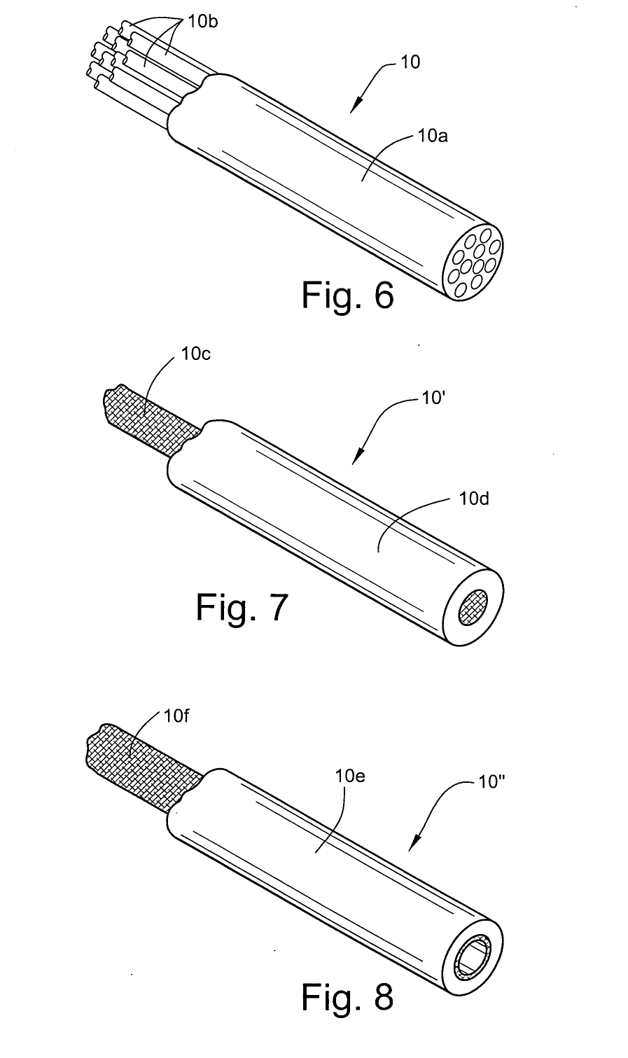

As shown in FIG. 6, the flexible composite connecting rod 10 (or 110 or 210) of the present invention may comprise a rod member 10a formed of a flexible plastic material having a predetermined compression strength, such as polyurethane, UHMW polyethylene, PEEK or Teflon, and a plurality of high tensile strength, low stretch, flexible reinforcing elements 10b extending longitudinally through the entire length of the rod member in substantially parallel relation. The reinforcing elements 10b may be slidable within apertures in the rod member 10a or may be bonded thereto in any suitable manner. Each of the reinforcing elements may be in the form of a cord, rope, braided tube or monofilament, woven or wire mesh strain relief device or the like formed of a suitable bio-compatible material, such as Kevlar, polyethylene, polyurethane, Teflon fiber, carbon fiber or stainless steel. The reinforcing elements 10b may be formed of any suitable elongated construction, other than a cord, rope, braid or mesh for serving the intended purpose in the flexible composite connecting rod 10 as hereinbefore described.

FIG. 7 illustrates a second embodiment of the flexible composite connecting rod 10′ (or 110 or 210) wherein a single large flexible reinforcing element 10c extends longitudinally through the entire length of the rod member 10d and is slidable within an aperture therein or is bonded thereto in any suitable manner. In this embodiment, the rod member 10d and flexible reinforcing element 10c may be formed of any construction or suitable materials as hereinbefore described.

FIG. 8 discloses a third embodiment of the flexible composite connecting rod 10″ (or 110 or 210) which comprises a rod member 10e and a tubular flexible reinforcing element 10f extending through the entire length thereof and slidably mounted within or connected thereto in any suitable manner. Within the scope of the present invention, the tubular element 10f may be disposed within a central elongated opening in the rod member 10e, or may be embedded in the central portion of a solid rod member. The rod member 10e and tubular reinforcing element 10f may be of any suitable construction or materials as hereinbefore described.

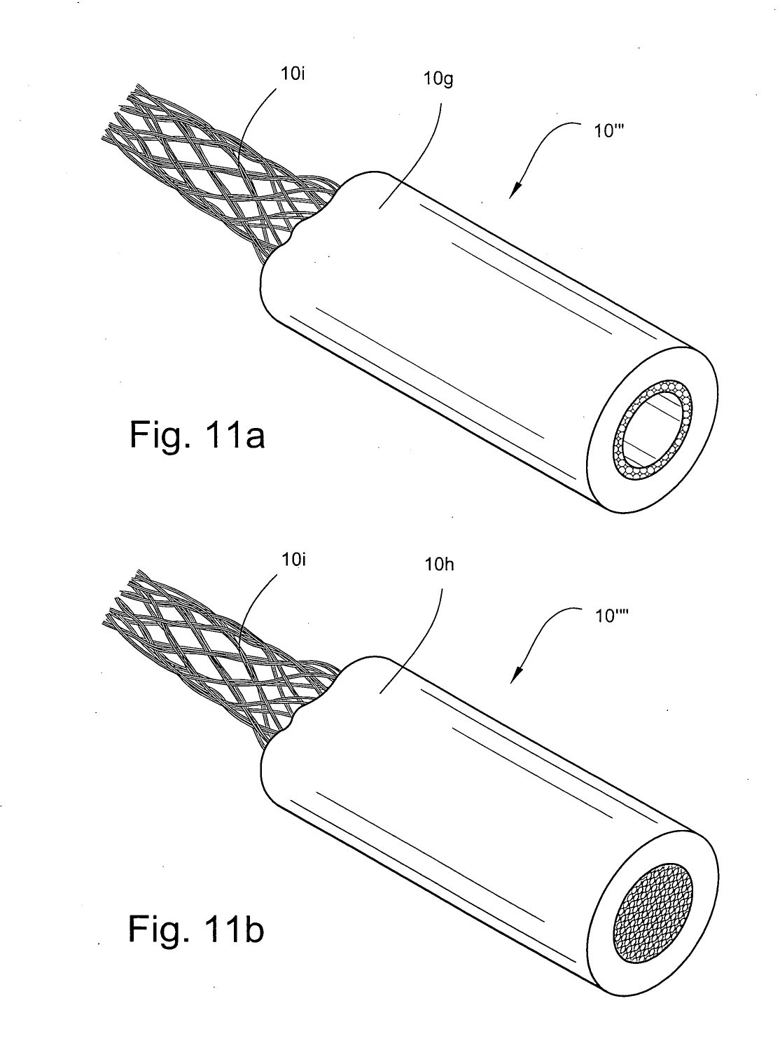

FIGS. 11a and 11b disclose fourth and fifth embodiments of the flexible composite connecting rod 10′″, 10″″ (or 110 or 210) which comprises a rod member 10g (FIG. 11a), 10h (FIG. 11b) and a tubular flexible reinforcing element 10i extending through the entire length thereof and slidably mounted within or connected thereto in any suitable manner. Within the scope of the present invention, the tubular element 10i may be disposed within a central elongated opening in the rod member 10g, as shown in FIG. 11a, or may be embedded in the central portion of a solid rod member 10h, as shown in FIG. 11b. The rod member 10g, 10h may be of any suitable construction or materials as hereinbefore described. The tubular reinforcing element 10i is a woven or wire mesh of any suitable material as hereinbefore described in the nature of a cable strain relief device, as shown in FIGS. 11a and 11b.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. Apparatus for connecting and stabilizing adjacent vertebral segments, comprising:
a flexible composite connecting rod extending between the segments; and
connection devices for connecting said rod to the vertebral segments;
said rod comprising a rod member formed of a flexible plastic material having a predetermined compression strength, and a high tensile strength, low stretch, flexible reinforcing element extending longitudinally through the entire length of said rod member.

2. The apparatus of claim 1 wherein said rod member has an aperture extending longitudinally therethrough, and said reinforcing element is slidable within said aperture.

3. The apparatus of claim 1 wherein said reinforcing element is bonded to said rod member.

4. The apparatus of claim 1, wherein said rod member is formed of polyurethane, UHMW polyethylene, PEEK or Teflon.

5. The apparatus of claim 4 wherein said reinforcing element is formed of Kevlar, polyethylene, polyurethane, Teflon fiber, carbon fiber or stainless steel.

6. The apparatus of claim 1, wherein said rod member is solid and said reinforcing element is a cord or rope extending through the middle portion thereof.

7. The apparatus of claim 6, wherein said reinforcing element comprises a plurality of cords or ropes extending in substantially parallel relation through said rod member.

8. The apparatus of claim 1, wherein said rod member is tubular with a central opening and said reinforcing element is a tubular cord, braid or woven or wire mesh strain relief device extending through the central opening of said rod member.

9. The apparatus of claim 1, wherein said rod member is solid and said reinforcing element is a tubular cord, braid or woven or wire mesh strain relief device extending through the central portion thereof.

10. The apparatus of claim 1, wherein each connection device is a pedicle screw having an opening in the head portion thereof for receiving and retaining said connecting rod therein.

11. The apparatus of claim 10, wherein said head portion comprises a set screw for applying pressure to said connecting rod to retain it therein.

12. The apparatus of claim 11, wherein a clamping pad is disposed between said set screw and said connecting rod, and the head portion of said pedicle screw comprises an inner shoulder or stop for engagement by the clamping pad to control the pressure on the connecting rod by the set screw.

13. The apparatus of claim 10, wherein ring members are mounted on the connecting rod and are received in the openings in the head portions of said pedicle screws.

14. The apparatus of claim 13, wherein each ring member is circular in cross section to provide for flexible alignment of the connecting rod on the pedicle screws.

15. The apparatus of claim 13, wherein each ring member is square or rectangular in cross section to provide for rigid alignment of the connecting rod on the pedicle screws.

16. The apparatus of claim 13 wherein said ring members are press fitted or crimped on said connecting rod in predetermined locations thereon.

17. The apparatus of claim 13 wherein said ring members are of split construction and are slidably mounted on said connecting rod.

18. A method for connecting and stabilizing adjacent vertebral segments, comprising:
providing a flexible composite connecting rod extending between the segments; and
connecting the end portions of the connecting rod to the vertebral segments;
said composite rod comprising a rod member formed of a plastic material having a predetermined compression strength, and a high tensile strength, low stretch, flexible reinforcing element extending longitudinally through the entire length of said rod member.

19. The method of claim 18, wherein said connecting rod is connected to the vertebral segments by polyaxial pedicle screws having head portions for receiving and retaining the end portions of said connecting rod therein.

20. The method of claim 18, wherein said rod member is solid and said reinforcing element is a cord extending through the middle portion thereof.

21. The method of claim 18, wherein said reinforcing element comprises a plurality of cords extending in substantially parallel relation through said rod member.

22. The method of claim 18, wherein said reinforcing element is a tubular cord, braid or woven or wire mesh strain relief device extending through a central portion of said rod member.

23. A composite connecting rod for connecting and stabilizing adjacent vertebral segments, comprising:
a rod member formed of a flexible plastic material having a predetermined compression strength; and
a high tensile strength, low stretch, flexible reinforcing element extending longitudinally through the entire length of said rod member.

24. The composite connecting rod of claim 23 wherein said rod member has an aperture extending longitudinally therethrough, and said reinforcing element is slidable within said aperture.

25. The composite connecting rod of claim 23 wherein said reinforcing element is bonded to said rod member.

26. The connecting rod of claim 23, wherein said rod member is formed of polyurethane, UHMW polyethylene, PEEK or Teflon.

27. The connecting rod of claim 26, wherein said reinforcing element is formed of Kevlar, polyethylene, polyurethane, Teflon fiber, carbon fiber or stainless steel.

28. The connecting rod of claim 23, wherein said rod member is solid and said reinforcing element is a cord, rope, braid, monofilament or woven or wire mesh strain relief device extending through the middle portion thereof.

29. The connecting rod of claim 28, wherein said reinforcing element comprises a plurality of cords, ropes, braids or monofilaments extending in substantially parallel relation through said rod member.

30. The connecting rod of claim 23, wherein said rod member is tubular and said reinforcing element is a tubular cord, rope, braid or woven or wire mesh strain relief device extending through the central opening thereof.

31. The connecting rod of claim 23, wherein said rod member is solid and said reinforcing element is a tubular cord, rope, braid or woven or wire mesh strain relief device extending through a central portion thereof.

32. The connecting rod of claim 28, wherein said reinforcing element comprises a tubular member.
